(19) United States
(12) Patent Application Publication
(10) Pub. No.: US 2024/0036003 A1
(43) Pub. Date: Feb. 1, 2024

(54) Method and System for Material Identification Using Magnetic Induction Tomography

(71) Applicant: NPL Management Limited, Teddington (GB)

(72) Inventors: Rafal Gartman, London (GB); Witold Chalupczak, Bracknell (GB)

(21) Appl. No.: 18/483,132

(22) Filed: Oct. 9, 2023

Related U.S. Application Data
(63) Continuation of application No. PCT/GB2022/050897, filed on Apr. 8, 2022.

(30) Foreign Application Priority Data
Apr. 9, 2021 (GB) 2105099.2

(51) Int. Cl. G01N 27/72; G01N 35/04

(52) U.S. Cl. CPC G01N 27/72; G01N 35/04

(57) ABSTRACT

Methods and apparatuses of identifying a type of material or material combination in a measurement location by Magnetic Induction Tomography (MIT) is disclosed. The methods can comprise providing a primary magnetic field primarily in a first direction into the measurement location; measuring an orthogonal component of a secondary magnetic field; classifying the orthogonal component of the secondary magnetic field with reference to a material or material combination type and thereby identifying a type of material or material combination in the measurement location. The orthogonal component of the secondary magnetic field is in a direction substantially orthogonal to the first direction.

### CROSS-REFERENCE TO RELATED APPLICATIONS

This application is a continuation of International Application PCT/GB2022/050897 having a filing date of Apr. 8, 2022, entitled “Method and System for Material Identification Using Magnetic Induction Tomography”, which is related to and claims priority benefits from GB patent application 2105099.2 filed on Apr. 9, 2021. This application also claims foreign priority benefits from the '099.2 application. The '897 and '099.2 applications are hereby incorporated by reference herein in their entireties.

### FIELD OF THE INVENTION

The present invention relates to methods and systems for material identification.

This work was funded by UK Department for Business, Energy and Industrial Strategy as part of the National Measurement System Program.

Magnetic Induction Tomography (MIT) measurements rely on the inductive coupling between a radio-frequency (rf) magnetic field, the so-called primary rf field, and the object of interests. See Griffiths, “Magnetic Induction Tomography”, Meas. Sci. Technol., 12, 1126-1131 (2001). See also Ma et al., “Magnetic Induction Tomography Methods and Applications: A Review”, Meas. Sci. Tech, 28, 072001-072012 (2017).

As a result of the coupling an object response is produced in the form of a secondary rf field. For objects whose response is dominated by electrical conductivity, eddy currents induced by the primary rf field produce the secondary rf field that opposes the driving field. This leads to dissipation of the primary rf field and reduced field penetration within the object. When the response is dominated by magnetic permeability the primary field creates within the object a magnetisation oscillating in phase with the driving field.

MIT provides a portfolio of measurements addressing a wide range of contemporary challenges in applied physics. In the area of non-destructive testing (NDT), inductive measurements enable detection of defects either covered by insulation or concealed within the object structure. See Auld et al., “Review of Advances In Quantitative Eddy Current Nondestructive Evaluation”, J. Nondestr. Eval. 18, 3-36 (1999); Perez et al., “Investigation in Detection of Fatigue Cracks Under Rivet Head Airframe Using Improved GMR Magnetometer In An Eddy Current System”, J. Elec. Eng., 55, 73-76 (2004); Deans et al., “Through-Barrier Electromagnetic Imaging with an Atomic Magnetometer”, Opt. Exp. 25, 17911-17917 (2017); Yoshimura et al., “Optimal Frequency of Low-Frequency Eddy-Current Testing for Detecting Defects on the Backside of Thick Steel Plates”, IEEE Transactions on Magnetics, 55, 8645817 (2019); Bevington et al, “Inductive Imaging of the Concealed Defects with Radio-Frequency Atomic Magnetometers”, Appl. Sci. 10, 6871 (2020).

Immediate applications of the technology lie in the energy sector where corrosion under insulation is responsible for a significant fraction of the losses in upstream segment. Implementations of MIT in object detection and surveillance include imaging through barriers and in turbulent underwater environments that prevent the use of visual of ultrasound technology.

The use of an rf atomic magnetometer as the sensor in MIT brings superior sensitivity. See Savukov et al, “Tunable Atomic Magnetometer for Detection of Radio-Frequency Magnetic Fields”, Phys. Rev. Lett. 95, 063004 (2005); Chalupczak et al, “Room Temperature Femtotesla Radio-Frequency Atomic Magnetometer”, Appl. Phys. Lett. 100, 242401 (2012); Deans et al., “Electromagnetic Induction Imaging with a Radio-Frequency Atomic Magnetometer”, Appl. Phys. Lett. 108, 103503 (2016); and Deans et al., “Sub-Picotesla Widely Tunable Atomic Magnetometer Operating at Room-Temperature in Unshielded Environments”, Rev. Sci. Instrum. 89, 083111 (2018).

The use of an rf atomic magnetometer as the sensor in MIT also brings a range of functionalities such as the ability to obtain vector measurements, high bandwidth in self-oscillating mode and tunability over a wide frequency range without compromise of performance. See Bevington et al, “Imaging of Material Defects with a Radio-Frequency Atomic Magnetometer”, Rev. Sci. Instrum. 90, 013103 (2019); Bevington et al, “Enhanced Material Defect Imaging with a Radio-Frequency Atomic Magnetometer”, J. Appl. Phys. 125, 094503 (2019). See also P Bevington et al, “Alkali-Metal Spin Maser for Non-Destructive Tests”, App. Phys. Lett. 115, 173502 (2019); and Bevington et al, “Magnetic Induction Tomography of Structural Defects with Alkali-Metal Spin Maser”, Appl. Opt. 59, 2276 (2020). See also Wickenbrock et al., “Eddy Current Imaging with an Atomic Radio-Frequency Magnetometer”, Appl. Phys. Lett. 108, 183507 (2016). See also Bevington et al., “Inductive Imaging of the Concealed Defects with Radio-Frequency Atomic Magnetometers”, Appl. Sci. 10, 6871 (2020).

Measurement of the object response with an rf atomic magnetometer relies on monitoring the change in the amplitude and phase of the rf resonance recorded with the magnetometer while scanning across the material.

Previous work has focused on the implementation of rf atomic magnetometers for inductive tomographic mapping in scenarios of defect detection and object surveillance. These include the demonstration of tomographic mapping of material thinning in steelwork, which represents the detection of corrosion under insulation. See Bevington et al, “Non-Destructive Structural Imaging of Steelwork with Atomic Magnetometers”, App. Phys. Lett. 113, 063503 (2018). The imaging provides us with a vector measurement (2D) of the secondary field, while the orientation of the sensor axis, i.e. direction of the bias magnetic field, defines which field components are being monitored. See Bevington et al, “Imaging of Material Defects with a Radio-Frequency Atomic Magnetometer”, Rev. Sci. Instrum. 90, 013103 (2019); Bevington et al, “Enhanced Material Defect Imaging with a Radio-Frequency Atomic Magnetometer”, J. Appl. Phys. 125, 094503 (2019).

With the sensor and the primary rf field coil axes parallel to each other the magnetometer signal represents only the amplitude of the secondary field which leads to increased image contrast. Implementation of a spin maser, an rf atomic magnetometer operating in self-oscillating mode, increases the data acquisition rate and makes the sensor immune to variations in the ambient magnetic field. See P Bevington et al, “Alkali-Metal Spin Maser for Non-Destructive Tests”, App. Phys. Lett. 115, 173502 (2019); and Bevington et al, “Magnetic Induction Tomography of Structural Defects with Alkali-Metal Spin Maser”, Appl. Opt. 59, 2276 (2020). The increase in measurement bandwidth comes at the price of the reduction of the part of the defect/object signature that results the phase matching condition in the sensor feedback loop. The application of a set of the primary rf field coils with opposite polarity, a dual frequency spin maser or an external phase scan can solve this issue. See Bevington et al, “Dual-Frequency Caesium Spin Maser”, Phys. Rev. A 102, 032804 (2020). Whilst this inductive tomographic mapping can provide information about the depth and spatial extent of a defect or object, it requires a scan of the sensor over the area of interest.

Aspects of the present invention seek to provide an improved method which can identify a type of material or material combination and system.

### SUMMARY OF THE INVENTION

According to an aspect of the invention, there is provided a method of identifying a type of material or material combination in a measurement location, comprising:
providing a primary magnetic field primarily in a first direction into the measurement location;
measuring an orthogonal component of a secondary magnetic field, the orthogonal component of the secondary magnetic field being in a direction substantially orthogonal to the first direction;
classifying the orthogonal component of the secondary magnetic field with reference to a material or material combination type and thereby identifying a type of material or material combination in the measurement location.

In embodiments, the type of material or material combination relates to the material nature, material composition or substance composition in the measurement location, for example of an object in the measurement location. In embodiments, identifying a type of material or material combination in the measurement location includes identifying a type of substance or combination of substances that an object in the measurement location is made from. A type of substance or combination of substances can be a particular substance or a particular combination of substances.

In embodiments, the type of material or material combination is independent of the shape and surface detail of the object.

In some embodiments, the type of material, material combination, substance, or substance combination can be defined by electrical conductivity and/or magnetic permeability.

In some embodiments, measuring the orthogonal component of the secondary magnetic field is performed with an anisotropic sensor configured to have lesser sensitivity to magnetic fields in the first direction.

In embodiments, the secondary magnetic field measured is that created as a response to the primary magnetic field by one or more objects, if present, in the measurement location. Of course, in some instances no object is present in the measurement location and therefore the measurement of the secondary magnetic field may be substantially zero, which can be used to identify the absence of a type of material.

In some embodiments, the anisotropic sensor and a magnetic field source for providing the primary magnetic field may be coaxial and optionally adjacent to the measurement location.

In some embodiments, the primary magnetic field oscillates at a frequency and measuring the orthogonal component of the secondary magnetic field includes measuring the orthogonal component of the secondary magnetic field at a plurality of frequencies of the primary magnetic field.

In some embodiments, the plurality of frequencies includes at least one frequency in at least two of a first, second and third frequency range, preferably at least one frequency in each of the first, second and third frequency ranges, the first frequency range preferably being no more than 4 kHz, the second frequency range preferably being from 4 kHz to 15 kHz, and the third frequency range preferably being above 15 kHz.

In some embodiments, measuring the orthogonal component of the secondary magnetic field includes measuring the orthogonal component of the secondary magnetic field for a plurality of angles of the first direction with respect to the measurement location.

In some embodiments, measuring the orthogonal component of the secondary magnetic field includes performing a spatial scan of the measurement location.

In some embodiments, performing the spatial scan of the measurement location includes moving a primary magnetic field source with respect to the measurement location and/or moving the measurement location with respect to a primary magnetic field source.

In some embodiments, performing the spatial scan of the measurement location includes detecting an edge and/or curvature of an object in the measurement location and preferably detecting a shape or partial shape of the object.

In some embodiments, classifying the orthogonal component of the secondary magnetic field includes identifying a contribution created by magnetisation and/or a contribution created by eddy currents.

In some embodiments, measuring the orthogonal component of the secondary magnetic field includes measuring an amplitude and optionally a phase thereof.

In some embodiments, measuring the orthogonal component of the secondary magnetic field is performed with an anisotropic sensor configured to have lesser sensitivity to magnetic fields in the first direction, wherein the anisotropic sensor is an atomic magnetometer configured to have lesser sensitivity to magnetic fields in the first direction.

In some embodiments, the atomic magnetometer is configured with a bias magnetic field axis along the first direction.

In some embodiments, classifying the orthogonal component of the secondary magnetic field includes comparing the orthogonal component of the secondary magnetic field to one or more reference standards and classifying based on a similarity to the one or more reference standards.

In some embodiments, the one or more reference standards is/are one or more reference images and/or one or more reference image fragments, and the similarity to the one or more reference standards is calculated using an inverse dependence on amplitude difference, preferably an inverse dependence on the square of amplitude difference.

In some embodiments, classifying the orthogonal component of the secondary magnetic field is performed by a machine learning algorithm trained on a plurality of reference standards, wherein the plurality of reference standards is a plurality of reference images and/or reference image fragments.

In some embodiments, the machine learning algorithm is configured to assist classification by controlling the method to selectively include one or more features recited above.

According to an aspect of the invention, there is provided a program configured to perform the classifying step when executed on a computing device as part of a method recited above.

According to an aspect of the invention, there is provided computer readable medium carrying the program recited above.

According to an aspect of the invention, there is provided a system comprising:
a computing device programmed with the program recited above;
a magnetic field source for providing the primary magnetic field; and
a or the magnetic field sensor.

The magnetic field sensor can be the anisotropic sensor recited above.

In some embodiments, the sensor is configured to provide measurements to the computing device.

According to an aspect of the invention, there is provided a system comprising:
a conveyor comprising a first section, a second section and a directional change between the first section and the second section, the conveyor being configured to convey objects from the first section to the second section via the directional change;
a magnetic field source for providing a primary magnetic field primarily in a first direction into a measurement location located at the directional change of the conveyor;
an anisotropic sensor configured to have lesser sensitivity to magnetic fields in the first direction and configured to measure an orthogonal component of a secondary magnetic field created by objects at the measurement location, the orthogonal component of the secondary magnetic field being in a direction substantially orthogonal to the first direction.

The system can include the computing device recited above.

Some embodiments can provide object content identification with magnetic inductive tomography, for example with a radio-frequency atomic magnetometer.

Some embodiments enable identification of metallic objects with additional information on possible composition (for example a distinction between aluminium and steel).

Some embodiments can provide fast screening with the option of more detailed imaging.

The inductive response of an object to an oscillating magnetic field reveals information about its electrical conductivity and magnetic permeability. Some embodiments of the invention can use a technique that uses measurements of the angular, frequency and spatial dependence of the inductive signal to determine object composition. Identification can be performed by referencing an object's inductive response to that of materials with mutually exclusive properties such as copper (high electric conductivity, negligible magnetic permeability) and ferrite (negligible electric conductivity, high magnetic permeability). The technique can use a sensor with anisotropic sensitivity to discriminate between the different characters of the eddy current and magnetisation driven object response.

### BRIEF DESCRIPTION OF THE DRAWINGS

Embodiments of the invention are described below, by way of example only, with reference to the accompanying drawings, in which:

FIG. 1 and FIG. 1A show generic components of a Magnetic Induction Tomography measurement setup according to an embodiment of the invention. Primary rf field 7 (green arrow) produced by the rf coil causes the inductive response of the object in the form of a secondary field 8 (yellow arrow). The signal is recorded by a sensor. Here, the radio-frequency atomic magnetometer as a sensor monitors only the components of the secondary field orthogonal to the sensor axis 5 (black arrow).

FIGS. 2A-2F depicts the secondary rf field produced by eddy currents (FIG. 2A, FIG. 2C, FIG. 2E, and FIG. 2F) and magnetisation (FIG. 2B and FIG. 2D) in measurement geometries where the normal to the object surface is either parallel (FIG. 2A and FIG. 2B) or tilted at an angle (FIG. 2C, FIG. 2D, FIG. 2E, and FIG. 2F) to the primary rf field direction. The red arrows 9 in FIG. 2B and FIG. 2D indicate how the lift off in each geometry is defined.

FIG. 3A and FIG. 3B depicts plots of the amplitude (FIG. 3A) and phase (FIG. 3B) of measured rf signals generated by scans over pairs of stainless steel, copper, and ferrite 35×35 mm² plates. Red dashed lines 10 mark the positions of the plates. For amplitude and phase images in the upper row the normal to the surface of the plates is parallel to the primary rf field, whilst there is a 150 tilt between them for images in the lower row. The images were recorded at operating frequency 29 kHz.

FIG. 4A and FIG. 4B shows amplitude (FIG. 4A) and phase (FIG. 4B) of the rf signal as a function of the angle between normal to object surface and the sensor axis recorded for the ferrite (dark blue points), stainless steel (light blue triangles), brass (green diamonds) and aluminium (red squares) plates. For reference the signal level recorded in absence of the sample is shown with solid black line 14. The measurement was performed at operating frequency 6.7 kHz.

FIG. 5A and FIG. 5B shows amplitude (FIG. 5A) and phase (FIG. 5B) of the rf signal as a function of the operating frequency recorded for the ferrite (dark blue points), stainless steel (light blue triangles), brass (green diamonds) and aluminium (red squares) plates. For reference the signal level recorded in the absence of the sample is shown with solid black line 15. The plates are tilted by 200 with respect to the primary rf field direction.

FIG. 6A, FIG. 6B and FIG. 6C show the measured change of the amplitude (FIG. 6A) and phase (FIG. 6B) of the rf signal recorded over the ferrite and aluminium 35×35 mm² plates at 3.5 kHz (red, labelled 11), 7 kHz (green, labelled 12), and 29 kHz (blue, labelled 13). FIG. 6C Image integrated over three frequencies for various types of plates.

FIG. 7A and FIG. 7B show an analysis of the object composition based on the signal amplitude integrated over the image area. The inductive images of the same plates as shown in FIG. 6, were recorded at 29 kHz. Each image (object) is represented by a point in 3D space defined by three orthogonal vectors that represent purely magnetically permeable (1,0,0-horizontal axis), electrically conductive (0,1,0—vertical axis) and background ‘no-object detected’ case (0,0,1-axis orthogonal to the plot plane). Position of the point shown in the plot is given by probabilities of the tested object having the reference properties (purely magnetically permeable, purely electrically conductive object, and no object detected). 7(a)/7(b) Distribution obtained with the custom metrics defined in the text/standard the deep learning model. Error bars correspond to standard deviation of the average value.

FIG. 7C shows schematics of examples used to create reference standards in some embodiments of the invention.

FIG. 8 shows the measured change of the amplitude (upper row) and phase (lower row) of the rf signal recorded over the ferrite and aluminium 35×35 mm² plates for series of operating frequencies (0.7 kHz, 1.4 kHz, 3.5 kHz, 7 kHz, 29 kHz).

All the measurements rely on monitoring the response of the atomic magnetometer while the frequency of the primary rf magnetic field is scanned through the rf resonance.

FIG. 9 shows the two quadrature components of the rf signal, (blue curve X and red curve Y, in both graphs the red curve being the curve that reaches a larger positive signal, are recorded by the lock-in referenced to the primary rf field frequency. Results are expressed in terms of the amplitude R=\sqrt{X²+Y²} and phase ϕ=arctan(Y/X).

### DETAILED DESCRIPTION OF ILLUSTRATIVE EMBODIMENT(S)

As mentioned in the introduction, with the sensor and the primary rf field coil axes of an atomic magnetometer assembly parallel to each other the magnetometer signal represents only the amplitude of the secondary field which leads to increased image contrast. Implementation of a spin maser, an rf atomic magnetometer operating in self-oscillating mode, can increase the data acquisition rate and make the sensor immune to variations in the ambient magnetic field. See P Bevington et al, “Alkali-Metal Spin Maser for Non-Destructive Tests”, App. Phys. Lett. 115, 173502 (2019); and Bevington et al, “Magnetic Induction Tomography of Structural Defects with Alkali-Metal Spin Maser”, Appl. Opt. 59, 2276 (2020). The increase in measurement bandwidth comes at the price of the reduction of the part of the defect/object signature that results the phase matching condition in the sensor feedback loop. The application of a set of the primary rf field coils with opposite polarity, a dual frequency spin maser or an external phase scan can solve this issue. See Bevington et al, “Dual-Frequency Caesium Spin Maser”, Phys. Rev. A 102, 032804 (2020). Whilst this inductive tomographic mapping can provide information about the depth and spatial extent of a defect or object, it requires a scan of the sensor over the area of interest. Although the scan time can be optimised there is a category of scenarios, such as security screening, that benefits from rapid measurements that are possible at a single location or does not require scanning of the whole object and determine whether more detailed screening is required. Usually, this decision is based on the ability to discriminate between different types of materials.

In general, any object shows some level of electric conductivity and magnetic permeability. The secondary rf field reflects the character of the dominating property, but the measured amplitude and phase of the inductive response depends on relative ratio between the electric conductivity and magnetic permeability, which in principle indicates the composition of the object.

In this disclosure, a technique is presented that can potentially help determine object composition and hence reduce measurement duration. In some embodiments it combines measurements of the angular, frequency and spatial dependence of the signal with comparisons of the object's inductive response to those of reference materials with mutually exclusive properties such as copper (high electric conductivity, negligible magnetic permeability) and ferrite (negligible electric conductivity, high magnetic permeability). While the rf signal frequency dependence has previously been shown to provide discrimination between different objects, the demonstration was limited to a narrow class of purely conductive materials and required a series of extra measurements for calibration See Wickenbrock. The discrimination discussed in Wickenbrock was based on the direct dependence of the inductive signal on the electrical conductivity of objects with negligible magnetic permeability. Identification of objects whose inductive response, the secondary rf field, results from both eddy currents and magnetisation components is more complex. Moreover, as demonstrated, the signal depends on the experiment geometry and the object shape, complicating both the measurements and data analysis. Frequency and angular dependence measurements are presented herein that are performed at single spot above the object, which can reduce the screening time. The disclosure provides validation of the technique, i.e. demonstration of a series of measurements that can provide discrimination between objects made of different materials. Analysis of the data for practical application can be improved by the introduction of various metrics, such as those based on machine learning. Two methods of inductive image analysis are demonstrated that can assist in identification of object composition, the first based on the signal amplitude's frequency dependence and the second using the amplitude integrated over the entire image area.

Embodiments of the invention exploit the observation that secondary magnetic fields created by magnetisation and eddy currents behave differently, enabling classification and identification of material or material combination types. In embodiments the material or material combination can also be described as the substance or substance combination that an object is made from.

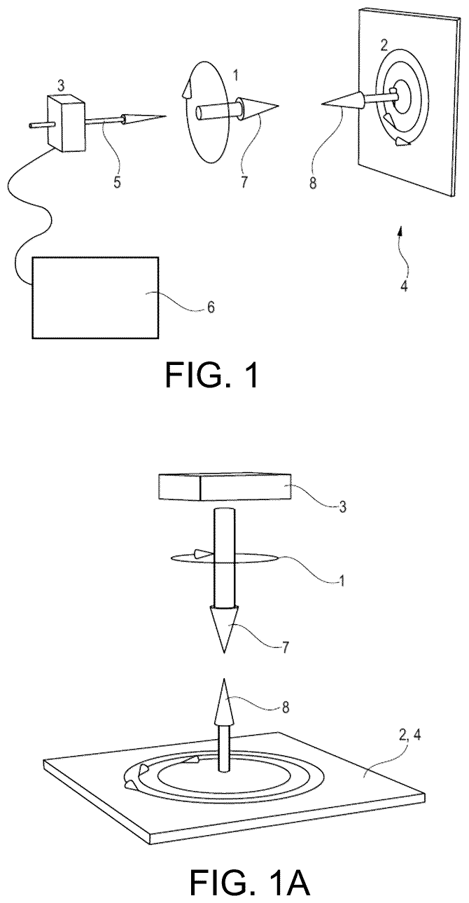

Embodiments of the invention provide a method of identifying a type of material or material combination in a measurement location. With reference to FIG. 1 and FIG. 1A, a system for the method includes a magnetic field source 1, in this embodiment an rf coil. The system is arranged to measure the contents of a measurement location 2. In FIG. 1 an object 4 shown as a plate is positioned in the measurement location, although the object is the item being measured and is therefore not necessarily itself a part of the system.

In the embodiments described herein, the system includes an anisotropic sensor 3 configured to have lesser or no sensitivity to magnetic fields in a first direction 5. In this embodiment, the sensor 3 is an rf atomic magnetometer with its axis in the first direction. In this embodiment, the first direction is from the sensor 3 towards the measurement location 2. In this embodiment, the anisotropic sensor 3 and the magnetic field source 1 are coaxial and adjacent to the measurement location with the magnetic field source 1 between the sensor and the measurement location; however, this arrangement is not necessarily the same in every embodiment.

In this embodiment, the system also includes a computing device 6.

The method includes providing an oscillating primary magnetic field 7, with the magnetic field source 1, primarily in the first direction 5 into the measurement location 2. This causes the object 4, where there is one in the measurement location 2, to create a secondary magnetic field 8, also referred to as an object response, for example as a result of eddy currents and/or magnetisation.

The rf atomic magnetometer is typically configured to operate at the frequency of the primary field. In order to achieve this, a bias field of the atomic magnetometer is configured to define a Larmor frequency at the frequency of the primary field.

The method includes measuring an orthogonal component of the secondary magnetic field 8, the orthogonal component of the secondary magnetic field being in a direction substantially orthogonal to the first direction 5. This is done advantageously with the anisotropic sensor 3 because of the lesser sensitivity of the sensor 3 in the first direction. Measuring the orthogonal component of the secondary magnetic field is advantageous because it can facilitate the identification of different behaviour of secondary magnetic fields created by magnetisation and eddy currents, leading to classification and identification of material or material combination types as discussed below. The signal represents amplitude and phase of the object response, although it is not necessary to use both in every embodiment, as discussed below.

As can be seen in FIG. 1, in this embodiment, the anisotropic sensor 3 is coupled to the computing device and is configured to provide measurements of the orthogonal component of the secondary magnetic field to the computing device 6. However, the computing device 6 is not necessary in every embodiment.

The method includes classifying the orthogonal component of the secondary magnetic field 8 with reference to a material or material combination type and thereby identifying a type of material or material combination in the measurement location 2, in the example of FIG. 1 a type of material or material combination of the object 4. In embodiments of the invention, material can also be described as substance and the material or material combination can also be described as the substance or substance combination the object is made from. The type of material or material combination can be a particular material, such as copper, aluminium, steel, ferrite, or a particular material combination, or it can be a type for example defined by electrical conductivity and/or magnetic permeability, and the type can relate to an individual material or a plurality of materials in combination, or it can be an absence of a type of material. The method can be used for identifying a concealed metallic object and optionally also its structure. In embodiments of the invention, the type of material or material combination is independent of object structure, shape and surface detail, but object structure can in some embodiments be identified in addition to the type of material or material combination.

The classifying step of the method is in some but not all embodiments performed by a program running on the computing device. For example the computing device can include a processor and a computer readable medium such as a memory, the program can be carried by the computer readable medium and the processor can configured to retrieve the program from the computer readable medium and execute it. It is to be noted that the computing device can be any kind of computing device which is able to receive the measurements by any means and able to perform the classifying step; it is not necessarily local to the rest of the system.

As explained in more detail below, measuring the orthogonal component of the secondary magnetic field 8 can be performed at a single spot of the measurement location 2, or over a spatial scan (2D map) of the measurement location 2. Performing the spatial scan of the measurement location 2 can include moving the primary magnetic field source 1 with respect to the measurement location 2 and/or moving the measurement location 2 with respect to a primary magnetic field source 1. It is to be noted that a spatial scan does not need to be a spatial scan of an entire object for reasons made clear below. Indeed, the measurement location may include only part of an object.

Measuring the orthogonal component of the secondary magnetic field 8 can include measuring an amplitude and optionally a phase thereof.

Different embodiments based on the system and method of FIG. 1, with or without the computing device, are discussed below, and explained with reference to experimental results.

As can be seen from the below, embodiments can use one or more of three types of relevant measurements:
Frequency scan: measurements at different Larmor frequencies
Angular scan: measurements with different object orientation
Spatial scan: recording of the MIT image, 2D map

Furthermore, object response can be referenced to predefined standards (such as response produced purely by eddy currents, magnetisation, empty space). Metrics can allow referencing to the standards.

Angular scans of the inductive response produced by eddy currents and magnetisation is different. Angular scans can therefore allow discrimination between materials.

In some embodiments, performing the spatial scan of the measurement location includes detecting an edge and/or curvature of an object in the measurement location and preferably detecting a shape or partial shape of the object.

As further illustrated below, in some embodiments a combination of the measurement geometry and spatial scans is analogous to the angular scan. This means that spatial scans may reveal not only the structure of the object but also its composition. Edges and curvatures of the objects rotate the secondary field in a way that mimics angular scans. Angular, frequency and spatial scans are not independent. In some embodiments they are coordinated. As explained below, machine learning software may in some embodiments decide the content and sequence of scans to identify the object composition.

The experimental measurements described herein were performed with the radio-frequency atomic magnetometer 3 operating in a magnetically unshielded environment. See Bevington et al, “Non-Destructive Structural Imaging of Steelwork with Atomic Magnetometers”, App. Phys. Lett. 113, 063503 (2018); Bevington et al, “Imaging of Material Defects with a Radio-Frequency Atomic Magnetometer”, Rev. Sci. Instrum. 90, 013103 (2019); and Bevington et al, “Enhanced Material Defect Imaging with a Radio-Frequency Atomic Magnetometer”, J. Appl. Phys. 125, 094503 (2019).

For the purposes of the techniques and embodiments described in this disclosure, the technical details of the atomic magnetometer 3 are not essential. A description of the sensor and instrumentation is presented elsewhere. See Bevington et al, “Imaging of Material Defects with a Radio-Frequency Atomic Magnetometer”, Rev. Sci. Instrum. 90, 013103 (2019; and Bevington et al, “Enhanced Material Defect Imaging with a Radio-Frequency Atomic Magnetometer”, J. Appl. Phys. 125, 094503 (2019).

Discussion of the sensor here is limited to the enumeration of its major components. Our rf atomic magnetometer instrumentation includes three major subsystems: lasers, caesium atomic vapour contained in a paraffin-coated cell and the detection. The cell is kept at ambient temperature (atomic density n_{Cs}=0.33×10¹¹ cm⁻³) in a static magnetic bias field, created by a set of nested, orthogonal, square Helmholtz coils. The strength of the bias field defines the operating (Larmor) frequency of the sensor. The laser system produces two beams. A circularly polarised pump beam, frequency stabilized to the 6 ²S_{1/2} F=3→6 ²P_{3/2} F′=2 transition (D2 line, 852 nm) propagates along the direction of the bias magnetic field. It creates a population imbalance within the ensemble of caesium atoms. A probe laser, whose frequency is tuned 2.75 GHz below the 6 ²S_{1/2} F=3→6 ²P_{3/2} F′=2 transition, propagates in a direction orthogonal to the pump beam. It monitors the atomic signal created by the coupling of the atoms and the rf magnetic fields (i.e. atomic coherence). The primary rf field, oscillating at the sensor operating frequency, is produced by the coil 1 located in the vicinity of the measurement location 2 containing the object 4. Lift off, the distance between the primary rf field coil 1 and the measurement location 2 or object 4, is between 2 mm and 20 mm, although this can be varied in other embodiments. The axis of the primary rf field is parallel to the bias field direction, that is in the first direction 5. It is important to stress that the atomic magnetometer can sense only the rf magnetic field that is perpendicular to the direction of the bias magnetic field. In the following the bias field direction as the axis of the sensor is referred to. The parallel orientation of the sensor axis and the primary rf field makes the sensor insensitive to the primary rf field. See Bevington et al, “Enhanced Material Defect Imaging with a Radio-Frequency Atomic Magnetometer”, J. Appl. Phys. 125, 094503 (2019). Consequently, the sensor readout, measured for example by a lock-in amplifier or recorded by a 2 MS/s data acquisition board, monitors directly the secondary rf field 8. This simplifies the analysis of the data and makes the normalisation procedure, essential in Wickenbrock, obsolete.

### Measurement Geometry

In some embodiments, measuring the orthogonal component of the secondary magnetic field 8 can include measuring the orthogonal component of the secondary magnetic field 8 for a plurality of angles of the first direction 5 with respect to the measurement location 2. This can be performed at a single point of the measurement location 2, or over a spatial scan of the measurement location 2. Note that, as mentioned above, a spatial scan of the measurement location 2 is not necessarily a spatial scan of an entire object, meaning that even with a spatial scan of the measurement location the outcome can be produced faster than a spatial scan of an entire object.

In such embodiments, measuring the orthogonal component of the secondary magnetic field preferably includes measuring the amplitude and phase.

In general, eddy currents and magnetisation induced within the object produce two secondary rf field components that have different amplitude and phase characteristics. Depending on the relative electric conductivity and magnetic permeability values, one of these components dominates the object's inductive response. In this section, it is shown that the measurement geometry, specifically the relative orientation between the normal to the object surface and the sensor axis, can suppress or enhance the contribution to the measurement signal from each component of the secondary field. This provides a mechanism to distinguish between them and classify the orthogonal component of the secondary magnetic field with reference to a material or material combination type to identify a type of material or material combination of the object. In particular, classifying the orthogonal component of the secondary magnetic field can include identifying a contribution created by magnetisation and/or a contribution created by eddy currents thereby to identify a type of material or material combination of the object.

Due to eddy current driven dissipation, penetration of the rf field within an electrically conductive object can be limited to a thin layer in the immediate vicinity of the surface. In the particular case of an object made from aluminium, the skin depth is 0.8 mm for a primary rf field frequency of 10 kHz. In general, it can be expected that for any object the component of the secondary field due to eddy currents is produced in the immediate vicinity of the surface and its direction is parallel to the normal to the surface. This means that the secondary field direction reflects the orientation of the surface and any change in the orientation of the object surface results in a change of the direction of the secondary field. This will manifest itself as a change in the detected rf signal amplitude and phase. In contrast, in objects with negligible electrical conductivity (and hence low rf field dissipation) and high magnetic permeability, such as ferrites, the direction of the secondary field is defined by magnetisation throughout the object. It is parallel to the primary rf field direction regardless of the orientation of the object. Hence, it can be expected that the component of the secondary field produced by magnetisation in any object mirrors the primary rf field direction.

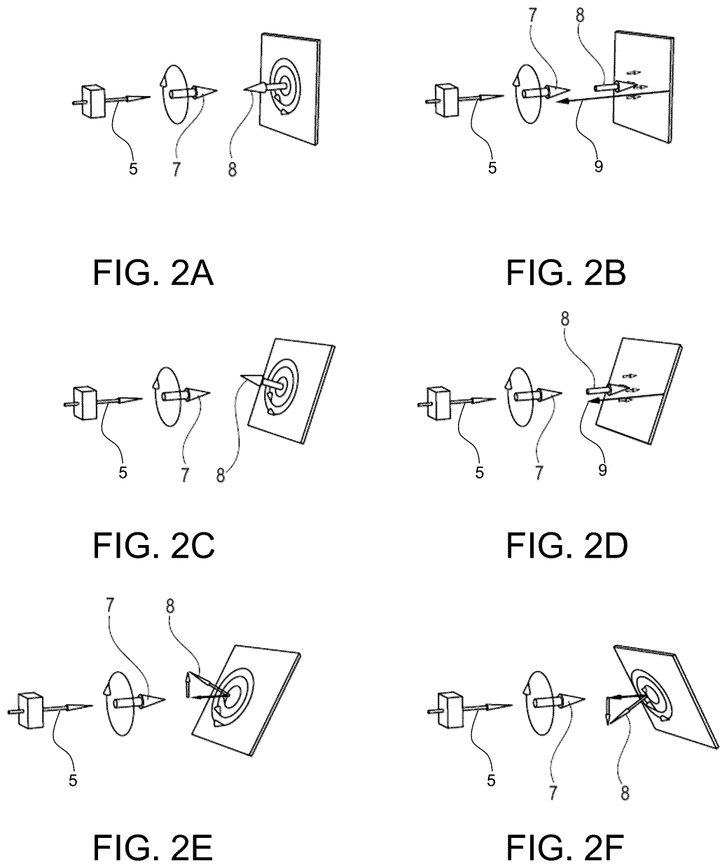

To gain further insight two measurement geometries are considered, the first where the normal to an object's surface is parallel to the primary rf field, FIG. 2A and FIG. 2B, and the second where there is a non-zero tilt between the two, FIG. 2C and FIG. 2D. As discussed earlier, the primary rf field direction 7 (green arrow) is parallel to the axis of the sensor (the first direction 5; black arrow). The atomic magnetometer 3 used as the sensor is insensitive to rf field components directed along this axis, so the primary rf field 7 does not contribute to the measured signal.

In the first configuration components of the secondary field 8 produced by both eddy currents and magnetisation are parallel to the axis of the sensor, and are in consequence invisible to it. With a non-zero tilt between the axes the direction of the component produced by eddy current is no longer parallel to sensor axis, making it visible to the sensor 3, FIG. 2C. The direction of the component produced by magnetisation remains parallel to the sensor axis, and so does not contribute to the detected signal, FIG. 2D. In general, with increasing angle between the sensor axis and the normal to the object surface the visibility of the eddy current driven component increases, while that of the magnetisation component doesn't change.

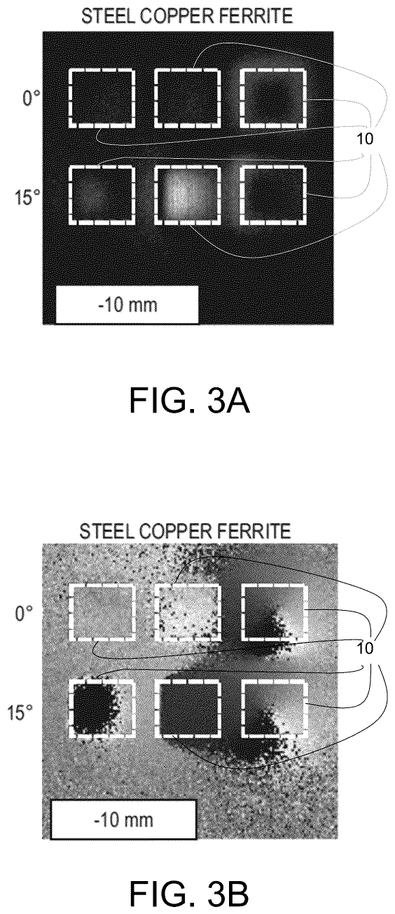

To illustrate the differences between object responses produced by eddy currents and magnetisation in different measurements geometries two sets of amplitude and phase inductive images were recorded. The images were generated by scans over three pairs of stainless steel, copper, and ferrite 35×35 mm² plates, marked with red dashed lines 10 in FIG. 3A and FIG. 3B. All the plates used in the experiment were 0.5 mm thick, except the ferrite, which was 2 mm thick. The images in FIG. 3A and FIG. 3B were recorded with the normal to the object surface either parallel to the primary rf field (upper row) or with a 15° tilt with respect to it (lower row).

The lift off, defined as shown in FIG. 2B as the distance from the primary rf coil to the axis of plate rotation, was 10 mm. The scans were performed at an operating frequency of 29 kHz. The choice of this particular frequency will be explained in the following section. The images of the ferrite plate represent the case when the inductive response is solely produced by the magnetisation of the object. Both ferrite amplitude images show a dark area produced by the centre of the plate surrounded by a bright square created by the edges. This results from the secondary field component parallel to the plate surface created by the plate edges Both ferrite phase images show the presence of a vortex, another signature of the plate edges. See Bevington et al, “Object Surveillance with Radio-Frequency Atomic Magnetometers”, Rev. Sci. Instrum. 91, 055002 (2020). For this material the amplitude and phase images recorded in different measurement geometries have the same structure and the signals have similar dynamic range, which supports the expectation that the magnetisation orientation is the same regardless of the measurement configuration. The smaller amplitude on right hand side of the image recorded with 15° tilt between the axes results from the bigger lift off.

The images of the copper plate represent the case when the inductive response is produced by eddy currents within the object. Images recorded in different geometries differ not only in amplitude but also in phase. The latter confirms that the direction of the secondary field produced by eddy currents depends on the orientation of the object's surface. It is worth pointing out the reversed character of the copper amplitude image recorded at 150 with respect to ferrite one. The inner part representing the secondary field created by the centre of the plate is bright and is surrounded by a dark square produced by the edges.

The stainless steel represents an object that exhibits both magnetic permeability and electrical conductivity. Because the permeability of stainless steel is smaller than that of the ferrite, the signature of the plate edges is small and neither the bright square nor the phase vortex is visible when the plate surface is not tilted.

With a 15° tilt between the sensor axis and the normal to the plate surface the amplitude and phase signatures become visible. Similarity of these signatures to those produced by the copper plate indicate that in this case the secondary field also originates from eddy currents.

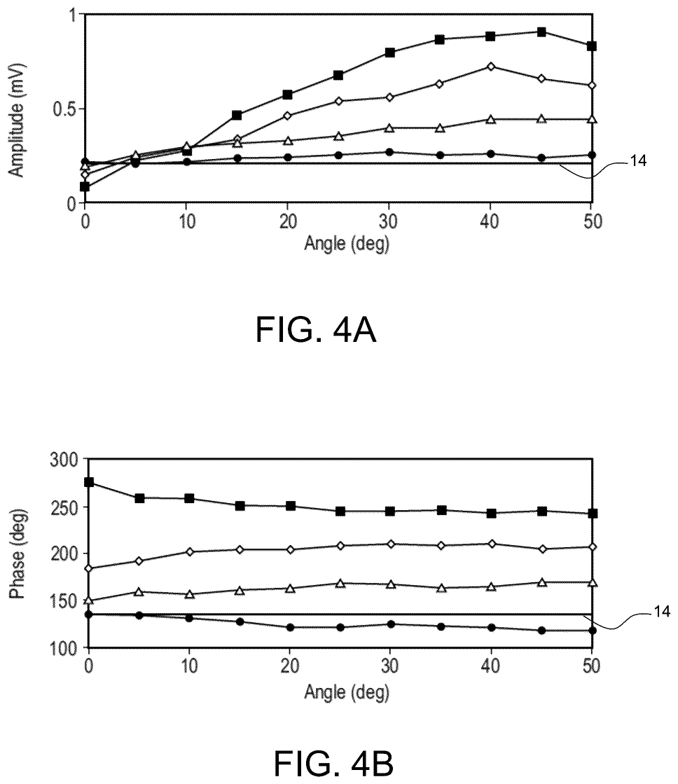

FIG. 4A and FIG. 4B shows the rf signal amplitude FIG. 4A and phase FIG. 4B as a function of the angle between the normal to the object surface and the sensor axis recorded at a single location above ferrite (dark blue points), stainless steel (light blue triangles), brass (green diamonds) and aluminium (red squares) plates. The measurement was done by placing the object on a support plate attached to a rotation mount, although in other embodiments the angle can be varied in other ways such as by a bend in a conveyor as described below. Care was taken to ensure that the primary rf field coil was located above the axis of rotation, as in FIG. 2, such that the change of object orientation does not affect its distance to the primary rf field coil.

The amplitude and phase of the signal produced by the ferrite plate does not change significantly with plate orientation, confirming that the secondary field generated by magnetisation mirrors the primary rf field direction. The high magnetic permeability and low electrical conductivity result in signals that are similar to those obtained in the absence of a sample.

It is worth reiterating that this is a result of the measurement configuration and magnetisation behaviour, FIG. 2B and FIG. 2D, where the sensor axis 5, marked with black arrow, is parallel to the direction of the primary rf field 7 (green arrow) and the secondary rf field 8 (yellow arrow).

In the case of the aluminium plate, the amplitude of the signal increases in angle range 0°-45°.

The secondary field 8 changes direction with plate rotation. The detected signal is sensitive only to the projection of the secondary field 8 onto the plane perpendicular to the sensor axis 5, with the amplitude given by the radius of this projected vector and the phase by the radial angle. Here the plate is rotated about an axis that is perpendicular to the sensor axis 5 (FIG. 2), which changes the radius of the projected vector but not the radial angle. As a result, a change in the amplitude of the signal is seen, but no change in phase.

In FIG. 2E and FIG. 2F show a conductive object, the sensor signal represents the orange component of the object response. An angular scan can cause a signal amplitude change. There can be a phase jump at 0°, i.e. where the primary field is orthogonal to object surface

The difference between the phases measured for ferrite and aluminium plates at any given tilt is about 1200 and reflects the different character of the effect that generates the secondary field. Steel plates possesses both significant electrical conductivity and magnetic permeability and while the latter dominates for low angles the former becomes visible with increasing angle. This is reflected in the increase of signal amplitude. The lower conductivity of the steel plate is reflected in the lower signal amplitude and phase relative to the aluminium plate when observed at larger tilt angles. The values of the amplitude and phase produced by the brass plate (between steel and aluminium) result from its conductivity value.

An angular dependence of the amplitude and phase of the signal similar to that presented in FIG. 4A and FIG. 4B is observed for operating frequencies above 4 kHz, which confirms that the effect requires eddy current generation limited to the immediate vicinity of the surface.

Because of the measurement configuration, where the sensor 3 has an insensitive axis that is aligned with the primary rf field 7, the angular dependence of the measured signal amplitude and phase is affected by an object's geometry. In the particular case of a plate the amplitude reaches a minimum for 0° and 900 when a surface of the plate faces the rf primary field 7, because the surface orthogonal to the primary rf field 7 does not contribute to the signal. This can be seen in FIG. 4A, where the signal amplitude reaches a maximum at 450 and starts to decrease for bigger angles. It is worth noticing that the thickness of the plate is not important. The same angular dependence of the signal amplitude would be observed in the case of a cubic box.

For regular shapes the number of minima in the signal amplitude and the angles at which they occur provide information about the symmetry of an object. In the more general case, a proper understanding of how the output of the local measurement depends on object geometry is important in the reconstruction of object shape and composition.

Frequency Scan

In some embodiments, measuring the orthogonal component of the secondary magnetic field 8 includes measuring the orthogonal component of the secondary magnetic field 8 at a plurality of frequencies of the primary magnetic field. This can for example be at a single point of the measurement location 2.

In such embodiments, measuring the orthogonal component of the secondary magnetic field preferably includes measuring the amplitude and phase.

In one embodiment, the plurality of frequencies includes at least one frequency in at least two of a first, second and third frequency range, in particular at least one frequency in each of the first, second and third frequency ranges, the first frequency range being no more than 4 kHz, the second frequency range being from 4 kHz to 15 kHz, and the third frequency range being above 15 kHz. In one embodiment, the plurality of frequencies includes 3.5 kHz, 7 kHz, and 29 kHz.

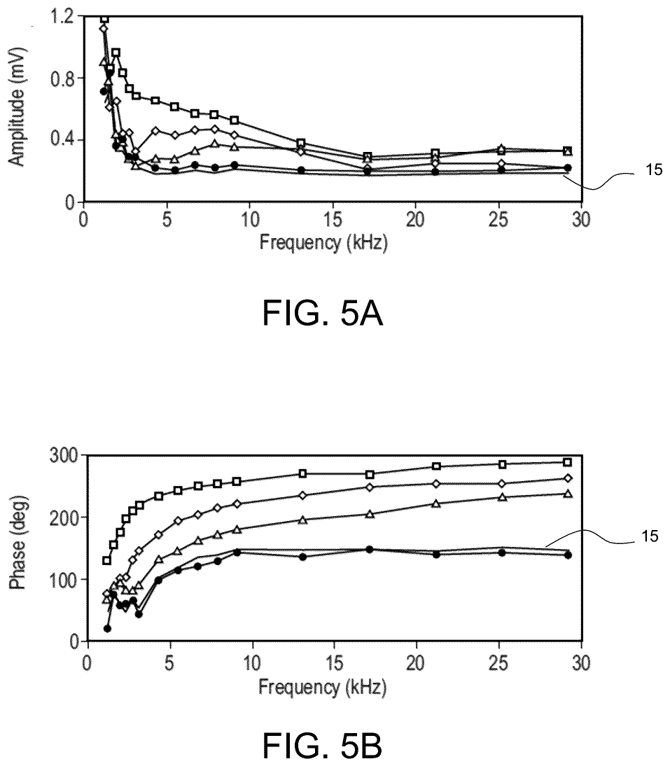

FIG. 5A and FIG. 5B show the amplitude and phase of the rf signal as a function of the rf field frequency for ferrite (dark blue points), stainless steel (light blue triangles), brass (green diamonds) and aluminium (red squares) plates. For reference the signal recorded in the absence of an object is also shown (solid black line 15). A 200 tilt between the normal to the plate surface and the primary rf field ensures that the component of the secondary field created by the eddy currents is visible.

Similarly, to the angle scan, the set of points representing the amplitude and phase of the signal observed over the ferrite plate overlaps with that observed in the absence of a sample. It is useful to take the data recorded over ferrite and aluminium plates as the points of reference. Analysis of the frequency dependencies of the steel and brass amplitude and phases relative to ferrite and aluminium indicates the presence of three frequency regimes. The first, up to 4 kHz, represents frequencies where low induction efficiency results in low eddy current density. In this range the amplitude of the steel overlaps with ferrite. For the frequencies in a second range, 4-15 kHz, a transition is observed in the steel signal amplitude and phase from the level observed over ferrite to that recorded over the aluminium. In the third frequency range, above 15 kHz, all observed amplitude and phase values are close to their asymptotic levels. It is worth pointing out that the 29 kHz operating frequency used to acquire the inductive images in FIG. 3 lies in this third frequency range, where the frequency dependence of the signals is negligible. It is worth comparing the frequency dependence of the phase changes of the signal generated by brass and steel objects. The phase measured with brass, although smaller in value, mirrors the dependence observed for aluminium across the whole frequency range. These phases changes indicate that the inductive properties are dominated by electrical conductivity. With stainless steel the phase behaviour is similar to that of the ferrite at low frequencies, but approaches that of aluminium at higher frequencies. This is consistent with an object that has significant magnetic permeability and electrical conductivity. Accordingly, as for the angle scan, classifying the orthogonal component of the secondary magnetic field can include identifying a contribution created by magnetisation and/or a contribution created by eddy currents thereby to identify a type of material or material combination of the object.

Object Mapping—Spatial Scan 1

A method of the inductive image analysis using the frequency dependence of the object response is presented.

In such embodiments, measuring the orthogonal component of the secondary magnetic field can include measuring the orthogonal component of the secondary magnetic field at a plurality of frequencies of the primary magnetic field as discussed in the previous section, but over a spatial scan of the measurement location.

It has been previously shown that for conductive objects an optimum value within a 1-2 kHz frequency range can be identified, which maximises the amplitude and contrast of features (defect, edge signatures) observed in the inductive images. See Bevington et al., “Inductive Imaging of the Concealed Defects with Radio-Frequency Atomic Magnetometers”, Appl. Sci. 10, 6871 (2020). Similar behaviour was seen in magnetically permeable objects, but where the optimum values were shifted to a higher frequency range. These observations suggest that monitoring the frequency dependence of the inductive image amplitude or contrast may indicate the object composition. The first analysis method follows the concept of colour perception by the human eye. White colour is a mixture of three basic (RGB) colours. Imbalance in these colour intensities will produce colour tones.

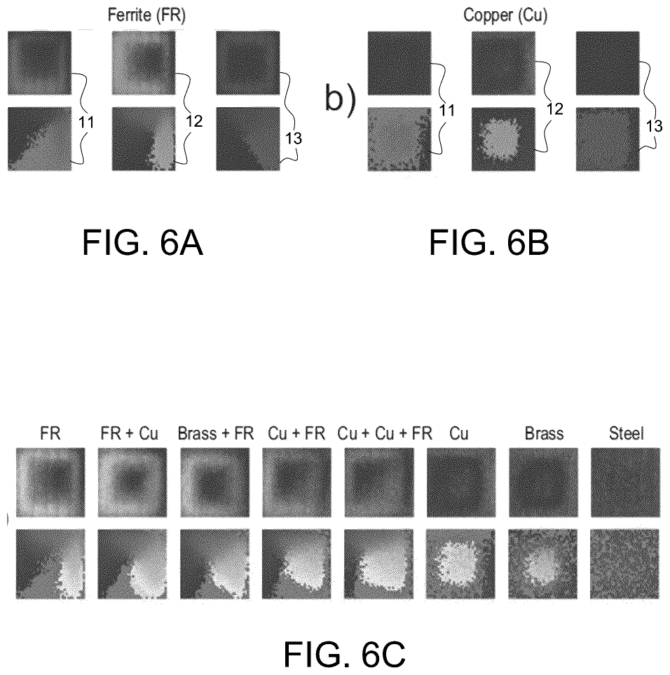

In order to explore this capability, images showing the amplitude and phase of the rf signal over either individual or stacks of 35×35 mm² plates made of various materials have been recorded. The images were recorded in a measurement configuration with the sensor axis parallel to the normal to the plate surface. In this configuration the non-zero signal is created solely by the edges of the object. See Bevington et al, “Object Surveillance with Radio-Frequency Atomic Magnetometers”, Rev. Sci. Instrum. 91, 055002 (2020). For each object (i.e. plate or ensemble of plates) a set of images at 3.5 kHz, 7 kHz, and 29 kHz were recorded and used them as the basis for an RGB representation. The images shown in FIG. 6A and FIG. 6B show scans over pure ferrite and copper plates recorded at 3.5 kHz (red, labelled 11), 7 kHz (green, labelled 12) and 29 kHz (blue, labelled 13). Each of the frequency values used in this measurement represents one of the three frequency ranges identified in the previous section. The images within each set were normalised to the maximum amplitude value recorded within the set and summed up.

FIG. 6C shows the images integrated over three frequencies (RGB representation) for various other plates and sets of plates. A simple visual analysis of the images can be done by taking the ferrite and copper plates as the reference points or reference standards and classifying based on a similarity to these reference standards. In this context, one can see the image of copper-ferrite plates set in FIG. 6C is a clear combination of the two references. Moreover, measuring two sheets of copper instead of one causes the conductivity fingerprint to be more pronounced, which is a manifestation of the layer's thickness. Reversing the order of the ferrite and copper layers does not significantly modify the structure of the image but does lead to a colour change.

Object Mapping—Spatial Scan 2

In some embodiments, the approach to inductive image analysis and therefore the classifying step of the method is based on the signal amplitude integrated over the image area. The method takes advantage of the opposite directions of the secondary fields created by the eddy currents and object magnetisation relative to an external reference such as a background field. Since the signal amplitude in the recorded image includes both the secondary and background fields, its total magnitude provides information on the relative orientation of these two components. Integration over an image area that includes elements like tilted surfaces, edges, etc. is in a sense analogous to the measurement of the angular dependence of the signal and can provide useful information for discrimination between object compositions.

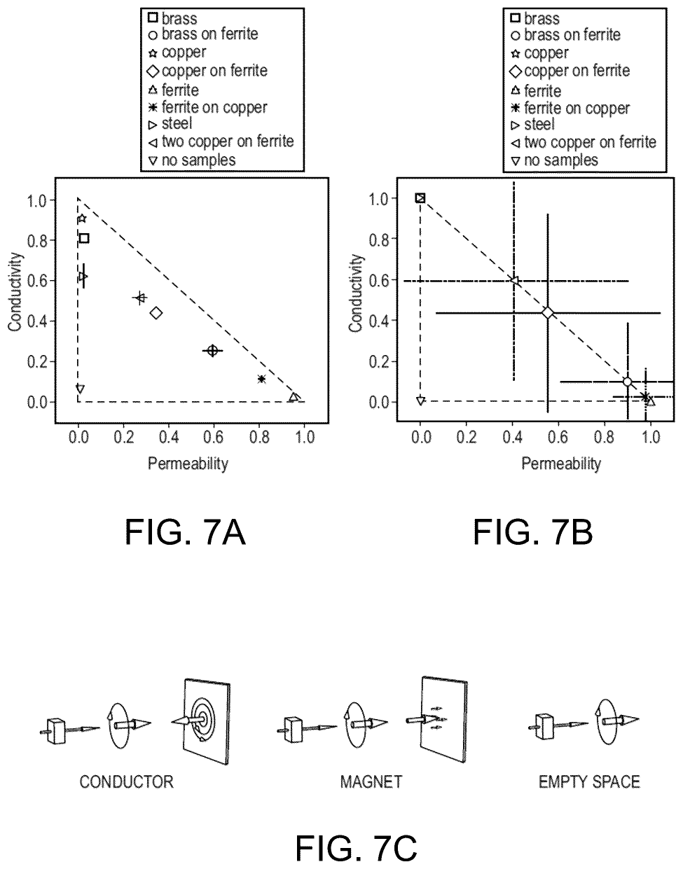

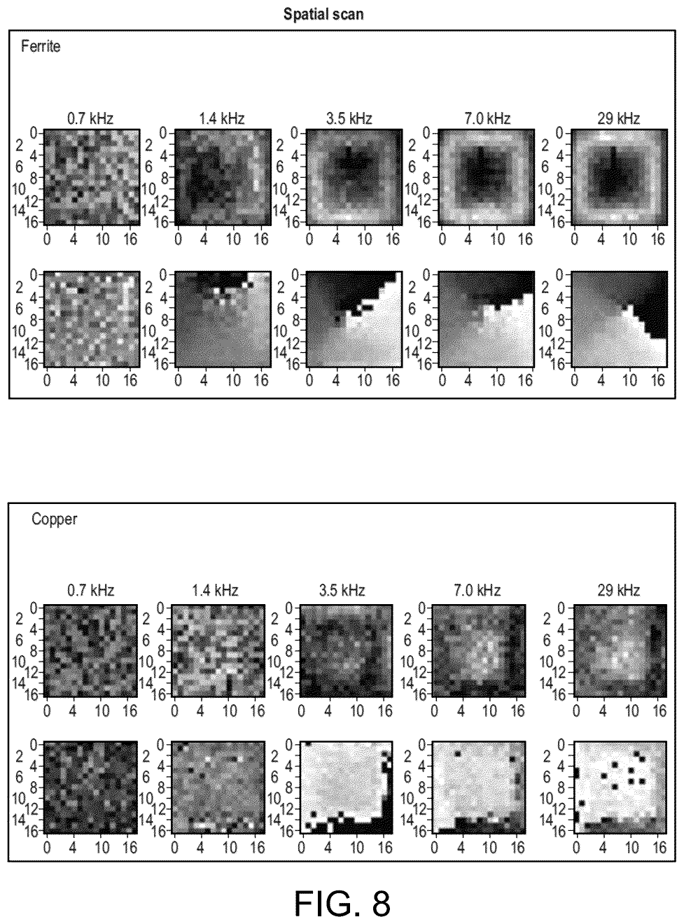

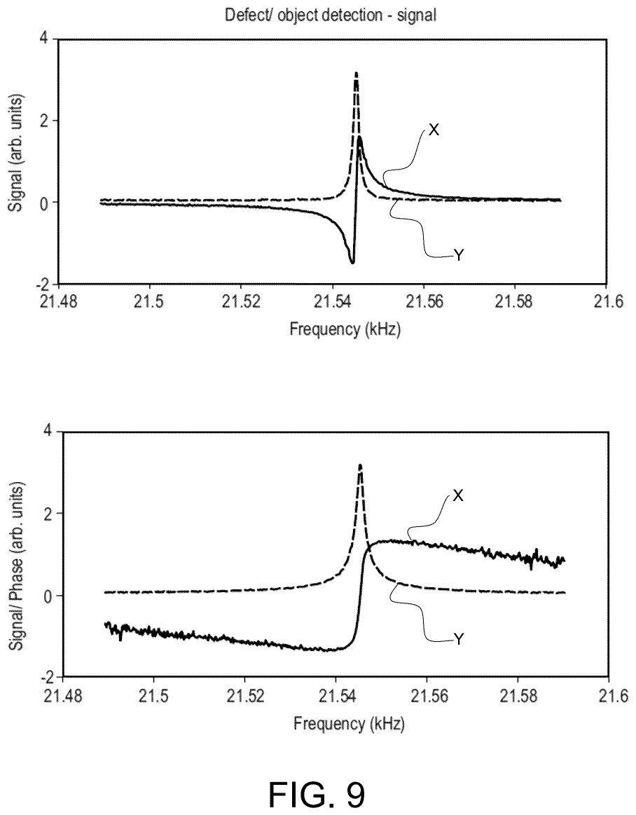

In this embodiment, classifying the orthogonal component of the secondary magnetic field includes comparing the orthogonal component of the secondary magnetic field to one or more reference standards in the form of reference images, and classifying based on a similarity to the one or more reference standards. In this embodiment, the classification includes evaluating a measure of the probability that the object properties are the same as those of three reference standards: ferrite (an approximation of a purely magnetic object), copper (purely conductive object) and the absence of the object (plate presence), although in other embodiments the choice of reference standards representing magnetic and conductive objects can be different. Schematic diagrams of the response of objects that can be used for the reference standards is shown in FIG. 7C The integrated image amplitudes of these references define three vectors of the orthogonal basis for 3D space. Location in this space of the point representing a tested object is identified by three coordinates specified by metrics (proximity) in respect to the three references. This location is a measure of the probability of seeing the object, and of the object being electrically conductive or magnetically permeable.

We introduce the metrics for classification, which are a measure of proximity, d, of the given data point (tested object) to the specific reference standard x, as: dₓ=1/Σᵢ₌₁^{N}(Rⁱ−Rₓⁱ)², where Rⁱ is the amplitude of a single pixel, i, in the tested object image, the tested object image being an image comprising measurements of the orthogonal component of the secondary magnetic field for a spatial scan over the measurement location 2, Rₓⁱ is the amplitude of the corresponding single pixel in the reference image, and N is the number of pixels in the image. The index x refers to either the purely magnetically permeable (1,0,0), purely electrically conductive (0,1,0) and no plate case (0,0,1). The signal amplitudes in the reference image, Rₓⁱ, are calculated as an average over 70 recorded images, although the number of images used to create a reference image can of course be different in other embodiments. Since the result, i.e. the data point location in 3D space represents probability, the sum of its coordinates is normalised, dₓ+d_{y}+d_{z}=1.

As can be seen, the similarity to the one or more reference standards is calculated using an inverse dependence on amplitude difference, in particular an inverse dependence on the square of amplitude difference.

It is worth discussing the metrics structure in more detail, particularly the choice of the inverse dependence on amplitude difference, (Rⁱ−Rₓⁱ)². The amplitude difference decreases as the object's properties become more similar to those of the reference. This leads to an increase in the value of the inverted factor, 1/(Rⁱ−Rₓⁱ)², which eventually becomes dominant over the other two factors, i.e. the proximity to the other two references. Normalisation of the sum of the coordinates and projection of its position on the xy (magnetic permeability vs electric conductivity) plane, places the measured point under the line x+y=1, in other words inside the triangle confined by x=0, y=0 and x+y=1. The smaller the integrated amplitude difference is, the higher its inverted value and the closer its normalised value approaches 1. Different types of metrics have been tested, in particular a linear dependence on integrated amplitude difference, as well as metrics including signal phase. While it has been verified that all metrics provide similar qualitative results, i.e. spatial distribution of the tested points relative to reference standards, and accordingly can be used in embodiments of the invention, the metrics described in detail above deliver the best differentiation between different materials.

FIG. 7A shows the location of the points representing different materials (plates, sets of plates) in the 2D electrical conductivity—magnetic permeability plane. This subspace is chosen as it enables us to demonstrate the discrimination of objects based on composition, in other words the classification/identification of material or material combination. As a result of the normalisation condition the distance from the origin reflects the probability that an object is present, and hence is a demonstration of our ability to detect objects. Each point in the plot is an average over 15 images, although of course in other embodiments a different number of images can be used to create a point. The points are grouped along the line connecting purely magnetic objects (1,0) and purely electrically conductive objects (0,1). This indicates all the tested samples showed a significant degree of conductivity or permeability. It is worth pointing out that the method can distinguish between the set of plates with ferrite on top of copper (blue square) and the same set in opposite order (yellow square).

The images used in the measurements shown were recorded at 29 kHz, but equivalent data taken at 7 kHz showed similar behaviour. Data recorded at 3.5 kHz were more scattered and led to poorer material discrimination, which is consistent with the observed weaker inductive signals at low frequencies, and accordingly frequencies above 4 kHz are preferably used. The similar distribution of the points in the data sets recorded at 7 kHz and 29 kHz indicates that even an image recorded at one frequency may contain enough information for the discrimination of object composition.

In another embodiment, classifying the orthogonal component of the secondary magnetic field is performed by a machine learning algorithm trained on a plurality of reference standards where the plurality of reference standards is a plurality of reference images and/or reference image fragments.

In particular, an analysis using a deep learning algorithm can be performed. We implemented an artificial neural network constructed from a combination of standard layers (2D convolution layers and fully connected layers) applied for computer vision tasks. The model was trained on around 2000 pictures, or reference standards, created by taking different fragments from 70 reference images, although different numbers of images can be used to create the reference standards in other embodiments. Similar fragments of scans were then used to test trained model predictions for unknown types samples. Averaged results for all samples are presented in FIG. 7B. Individual points are strongly scattered which resulted in bigger uncertainties than in FIG. 7A but the distributions of points are similar. The advantage of the algorithm is its ability to make a decision based on the fragments of the whole sample.

The ability to discriminate between ferrite plates and a mixture of copper and ferrite plates (FIG. 7) shows that the combination of the measurement geometry and the difference in angular responses generated by eddy currents and magnetisation (FIG. 2) allows us to see objects behind barriers or within electrically conductive enclosures.

An important concern is the practicality of implementing the presented methods in object screening. The results shown in FIG. 7 were recorded with objects that have the same geometry and dimensions, which is a highly idealized case. It has been verified that similar results to these presented in FIG. 7 can be obtained from sections of images that contain generic features such as flat surfaces and edges. This facilitates the use of a geometry non-specific procedure that combines large-scale inductive imaging of an object followed by the identification of appropriate features for composition analysis. The challenge of comparing results from objects with different geometries can also be addressed by more powerful tools such as supervised and unsupervised machine learning methods, which have proven to be very successful tools in solving similar problems. The implementation of machine learning used to generate the results in FIG. 7B was successful despite using only the amplitude of the measured signals at a single frequency. Enhanced performance would be expected from an implementation incorporating a combination of frequency, spatial and angle data.

In other words, in some embodiments of the invention, measuring the orthogonal component of the secondary magnetic field can include measuring the orthogonal component of the secondary magnetic field at a plurality of frequencies of the primary magnetic field, measuring the orthogonal component of the secondary magnetic field for a plurality of angles of the first direction with respect to the measurement location and/or performing a spatial scan over the measurement location, in any of the manners discussed above. In addition, using the measurements from any of these, classifying the orthogonal component of the secondary magnetic field can include comparing the orthogonal component of the secondary magnetic field to one or more reference standards and classifying based on a similarity to the one or more reference standards and/or classifying the orthogonal component of the secondary magnetic field can be performed by a machine learning algorithm trained on a plurality of reference standards where the plurality of reference standards is a plurality of reference images and/or reference image fragments. The classification can be implemented as discussed above, but the reference standards would be selected in accordance with the measurements it is intended to work with.

It has been demonstrated that a combination of the three degrees of freedom (spatial, angular and frequency) applied in the inductive measurements can provide sufficient information to deduce object composition (although, as explained above, not all three degrees of freedom need to be applied in every embodiment). It has been anticipated that similar information combined with advanced machine learning techniques will provide an even more versatile and effective tool, in which an optimised measurement sequence for a specific implementation is determined by the machine controlling the process. In this scenario the actual test consists of a series of moves using some or preferably all the degrees of freedom in a sequence that is autonomously decided and continuously updated by a pretrained machine learning model that, at the end of the measurement procedure, would provide some specific information about the interrogated object based on the collected data.

In other words, in further embodiments, the machine learning algorithm is configured to assist classification by controlling the method to selectively include the features of one or more of: measuring the orthogonal component of the secondary magnetic field at a plurality of frequencies of the primary magnetic field, measuring the orthogonal component of the secondary magnetic field for a plurality of angles of the first direction with respect to the measurement location and/or performing a spatial scan over the measurement location, in any of the manners discussed above.

In conclusion, it has been demonstrated that a series of MIT measurements can assist in the identification of an object's composition. It has been shown that the angular, frequency and spatial dependencies of the rf signal recorded over the objects can discriminate between object made of materials with different magnetic permeability and electrical conductivity. The discrimination is made possible through the use of a sensor with an insensitive axis. This eliminates the contribution of the primary rf field to the signal and gives the sensor a different sensitivity to the eddy current and magnetisation components of an induced response. The influence of object shape on the angular dependence of the rf signal have been discussed. Whilst frequency scans can be performed at a single location over an object, the measurement of angular dependence requires a physical change of the measurement configuration. This can be performed in various ways. In the specific case of goods screening, the objects are often transferred with a conveyor such as a conveyer belt, from a first section of the conveyor to a second section of the conveyor via a directional change of the conveyor, such as a bend, between the first and second sections. Location of the measurement location and sensor at the directional change, or bend, allows the measurement of objects at similar distances but at different orientations relative to the sensor axis.

Finally, it has been demonstrated that even very simple methods for acquiring and analysing inductive images can successfully discriminate between different materials.

As indicated above, applications of embodiments can include screening of goods/luggage possibly in on-line process, with fast measurements flagging up potential suspicious content. Security, border checks, airport luggage screening, weapons detection, materials characterisation/quality control, underground excavations and homeland security are suitable applications.

Although the atomic magnetometer in the above described embodiments is made to have lesser sensitivity to magnetic fields in the first direction by arranging its bias field direction along the first direction, this can be achieved in other ways in other embodiments, for which reference is made to WO 2020/051953, which is incorporated herein by reference in its entirety. Moreover, an atomic magnetometer is not needed in every embodiment; other sensors can be used, preferably anisotropic sensors which can measure the orthogonal component of the secondary magnetic field and be configured to have lesser sensitivity to magnetic fields in the first direction.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

What is claimed:

1. A method of identifying a type of material or material combination in a measurement location, comprising:
providing a primary magnetic field primarily in a first direction into the measurement location;
measuring an orthogonal component of a secondary magnetic field, the orthogonal component of the secondary magnetic field being in a direction substantially orthogonal to the first direction; and
classifying the orthogonal component of the secondary magnetic field with reference to a material or material combination type and thereby identifying a type of material or material combination in the measurement location.

2. The method of claim 1, wherein measuring the orthogonal component of the secondary magnetic field is performed with an anisotropic sensor configured to have lesser sensitivity to magnetic fields in the first direction.

3. The method of claim 1, wherein the primary magnetic field oscillates at a frequency and wherein measuring the orthogonal component of the secondary magnetic field includes measuring the orthogonal component of the secondary magnetic field at a plurality of frequencies of the primary magnetic field.

4. The method of claim 3, wherein the plurality of frequencies includes at least one frequency in at least two of a first, second and third frequency range, preferably at least one frequency in each of the first, second and third frequency ranges, the first frequency range preferably being no more than 4 kHz, the second frequency range preferably being from 4 kHz to 15 kHz, and the third frequency range preferably being above 15 kHz.

5. The method of claim 1, wherein measuring the orthogonal component of the secondary magnetic field includes measuring the orthogonal component of the secondary magnetic field for a plurality of angles of the first direction with respect to the measurement location.

6. The method of claim 1, wherein measuring the orthogonal component of the secondary magnetic field includes performing a spatial scan of the measurement location.

7. The method of claim 6, wherein performing the spatial scan of the measurement location includes moving a primary magnetic field source with respect to the measurement location and/or moving the measurement location with respect to a primary magnetic field source.

8. The method of claim 6, wherein performing the spatial scan of the measurement location includes detecting an edge and/or curvature of an object in the measurement location and preferably detecting a shape or partial shape of the object.

9. The method of claim 1, wherein classifying the orthogonal component of the secondary magnetic field includes identifying a contribution created by magnetisation and/or a contribution created by eddy currents.

10. The method of claim 1, wherein measuring the orthogonal component of the secondary magnetic field includes measuring an amplitude and optionally a phase thereof.

11. The method of claim 1, wherein measuring the orthogonal component of the secondary magnetic field is performed with an anisotropic sensor configured to have lesser sensitivity to magnetic fields in the first direction, wherein the anisotropic sensor is an atomic magnetometer configured to have lesser sensitivity to magnetic fields in the first direction.

12. The method of claim 11, wherein the atomic magnetometer is configured with a bias magnetic field axis along the first direction.

13. The method of claim 1, wherein classifying the orthogonal component of the secondary magnetic field includes comparing the orthogonal component of the secondary magnetic field to an at least one reference standard and classifying based on a similarity to said at least one reference standard.

14. The method of claim 13, wherein said at least one reference standard is one or more reference images and/or one or more reference image fragments, and the similarity to said at least one reference standard is calculated using an inverse dependence on amplitude difference, preferably an inverse dependence on the square of amplitude difference.

15. The method of claim 1, wherein classifying the orthogonal component of the secondary magnetic field is performed by a machine learning algorithm trained on a plurality of reference standards, wherein the plurality of reference standards is a plurality of reference images and/or reference image fragments.

16. A program configured to perform, when executed on a computing device as part of a method according to claim 1, a step of classifying the orthogonal component of the secondary magnetic field with reference to a material or material combination type and thereby identifying a type of material or material combination in the measurement location.

17. Computer readable medium carrying the program of claim 16.

18. A system comprising:
said computing device programmed with the program of claim 16;
a magnetic field source for providing the primary magnetic field; and
a magnetic field sensor.

19. The system of claim 18, wherein the magnetic field sensor is configured to provide measurements to the computing device.

20. A system comprising:
a conveyor comprising a first section, a second section and a directional change between the first section and the second section, the conveyor being configured to convey objects from the first section to the second section via the directional change;
a magnetic field source for providing a primary magnetic field primarily in a first direction into a measurement location located at the directional change of the conveyor; and
an anisotropic sensor configured to have lesser sensitivity to magnetic fields in the first direction and configured to measure an orthogonal component of a secondary magnetic field created by objects at the measurement location, the orthogonal component of the secondary magnetic field being in a direction substantially orthogonal to the first direction.
